(19) United States
(12) Patent Application Publication
(10) Pub. No.: US 2023/0330004 A1
(43) Pub. Date: Oct. 19, 2023

(54) COMPOSITION COMPRISING GINSENG EXTRACT FOR REFORMING SKIN MICROBIOTA

(71) Applicant: KOREA GINSENG CORP., Daejeon (KR)

(72) Inventors: Joon Hyuk HOU, Daejeon (KR); Seung Ho SO, Daejeon (KR); Seung Ho LEE, Daejeon (KR)

(73) Assignee: KOREA GINSENG CORP., Daejeon (KR)

(21) Appl. No.: 18/042,731

(22) PCT Filed: Aug. 9, 2021
(86) PCT No.: PCT/KR2021/010519

§ 371 Date: Feb. 23, 2023

(30) Foreign Application Priority Data
Aug. 27, 2020 (KR) 10-2020-0108712

(51) Int. Cl. A61K 8/9789; A61Q 17/00; A61K 8/34

(52) U.S. Cl. CPC A61K 8/9789; A61Q 17/005; A61K 8/345

(57) ABSTRACT

Provided is a composition for reforming skin microbiota comprising a ginseng extract; a ginseng-derived antibacterial agent; and alkanediol, to increase the fraction of beneficial bacteria in skin microbiota by promoting the growth of beneficial bacteria and inhibiting the growth of harmful bacteria, whereby the constitution of skin microbiota is reformed and maintained in a balanced state, ultimately helping improve human skin health.

### TECHNICAL FIELD

The present invention relates to a composition for reforming skin microbial flora, comprising a ginseng extract.

### BACKGROUND ART

Microbiome is a compound word from a combination of the words microbiota and genome, and refers to the community of microorganisms including the totality of microorganisms residing or co-existing in all environments such as humans, animals and plants, soils, ocean, lakes, rocks, atmospheres, etc. and their genetic material. Recently, a microbiome is also used to refer to the community of microorganisms in various environments. Various microbiomes have been investigated, such as plant microbiomes, animal microbiomes, environmental microbiomes, human microbiomes, gut microbiomes, skin microbiomes, and the like. Among such microbiomes, human microbiomes in particular have been most actively investigated.

The human body is a complex and active ecosystem, and there are various microorganisms in the gut, skin, etc. in the human body. There are 500 to 1,000 different types of microorganisms symbiotically residing inside the gut, skin, etc., forming microbial flora. Gut microorganisms found in the gut synthesize substances such as vitamins, and supply them to the human body, and also play a role in preventing infections caused by pathogenic microorganisms introduced through contaminated food.

Microbial flora resident on the skin surfaces influences a person's physiological functions, such as growth, nutrition, immunity, and resistance to pathogens, while also acting as a barrier that prevents invasions of foreign microorganisms, in particular, pathogenic microorganisms. Also, the resident microbial flora of the skin plays a role in adjusting the pH of skin and acts as a skin barrier, and is also known to influence the state of the skin through changes in skin lipid content.

The skin microbial flora includes, not only beneficial microbes, but also harmful microbes that are harmful to the human body. Therefore, for healthy skin, there is a need to promote the growth of beneficial microbes and inhibit the growth of harmful microbes.

Panax ginseng C.A. Meyer, which is a perennial herb of the genus Panax and family Araliaceae, is traditionally used in the Oriental medicine, has a sweet and bitter taste, exhibits the warm property, and is efficacious as herbal medicine in replenishing the body's energy, protecting the spleen, strengthening the lungs, and the like.

The main bioactive substances of ginseng include saponin, which is also known as ginsenosides, and essential oil components, polyacetylene, phenol components, glycosides, acidic peptides, etc. and also include other components such as vitamins, sugars, and minerals. Effects on the central nervous system, enhancement of immune functions, and anti-cancer effects, etc. have been reported as general bioactive effects of ginseng. In particular, approximately 40 types of ginsenosides were discovered thus far, and such ginsenosides were found to have a wide range of effects in the endocrine system, the immune system, and the metabolic system as well as the central nervous system, and as such, have demonstrated desirable effects in terms of regulation of bodily functions, that is, the normalization of physiological functions. These ginsenosides may have activities similar to one another or activities that oppose each other, and it is known that a particular ginsenoside alone, or ginsenosides of multiple types together through interactions, achieve various effects.

The present inventors found that a ginseng extract promotes the growth of beneficial microbes and discovered a potential use of the ginseng extract as prebiotics for skin. In this context, the present inventors investigated into compositions capable of selectively increasing beneficial microbes alone while inhibiting the growth of harmful microbes, and as a result, discovered that using a ginseng extract in combination with a ginseng-derived antibacterial agent and hexanediol can efficiently inhibit the growth of harmful microbes while promoting the growth of beneficial microbes, thus arriving at the present invention.

### DISCLOSURE OF THE INVENTION

### Technical Problem

The objective of the present invention is to provide a composition for reforming skin microbial flora, which can promote the growth of beneficial microbes in skin microbial flora while inhibiting the growth of harmful microbes.

### Technical Solution

One aspect of the present invention provides a composition for reforming skin microbial flora, the composition including: a ginseng extract; a ginseng-derived antibacterial agent; and an alkanediol.

### Advantageous Effects

The composition for reforming skin microbial flora of the present invention may increase the proportion of beneficial microbes in skin microbial flora by promoting the growth of beneficial microbes while inhibiting the growth of harmful microbes. Accordingly, the composition of the present invention may reform the composition of skin microbial flora and maintain the same in a balanced state, and as such may ultimately provide a benefit in improving the health of human skin.

Specifically, the composition of the present invention may show an effect of delaying the speed of skin aging by increasing the proportion of beneficial microbes in skin microbial flora, or alleviating a skin condition by reducing the proportion of harmful microbes that adversely affect the skin.

Further, the composition for reforming skin microbial flora of the present invention includes a ginseng concentrate and a ginseng-derived antibacterial agent as main ingredients, and since an alkanediol, even when used in a low concentration, exhibits a desirable effect of promoting the growth of beneficial microbes versus harmful microbes, there are advantages that the composition may show a low toxicity, cause less irritations to skin, and have a desirable stability.

### BRIEF DESCRIPTION OF THE DRAWINGS

FIG. 1 is a graph comparing fructooligosaccharides and a red ginseng concentrate in terms of the effect of promoting the growth of beneficial skin microbes.

### MODE FOR CARRYING OUT THE INVENTION

Herein below, the present invention is described in greater detail.

The terms and words used in the present specification and claims should not be limited to their accustomed and dictionary meaning, but rather, should be interpreted to have the meaning and concept consistent with the technical spirit of the present invention, in light of the principle that the inventor can suitably define the terms in the way that best describes the invention.

In the present invention, the term ‘microbial flora’ refers to a community of microorganisms including bacteria, fungi, and other prokaryotic organisms found in the human body, generally inhabiting the gut, skin, or the like.

In the present invention, the term ‘skin microbial flora’ refers to a community of microorganisms found in the skin, most of which inhabiting the skin surfaces such as scalp, pores, and the like. The skin microbial flora includes beneficial skin microbes that exhibit effects beneficial to skin, such as retaining water in the skin, eliminating reactive oxygen species, etc. and also includes harmful skin microbes that cause skin troubles such as acne and abscesses.

Examples of the beneficial skin microbes include, but are not limited to, Staphylococcus sp. strains, e.g., Staphylococcus epidermidis, Streptococcus sp. strains, e.g., Streptococcus pneumonia, and Lactobacillus sp. strains, e.g., Lactobacillus acidophilus, Lactobacillus Rhamnosus, and Bifidobacterium bifidum, and the like.

In particular, Staphylococcus epidermidis is a gram-positive bacterium commonly found on the skin, mucous membranes, etc. in human and animals. It is known that Staphylococcus epidermidis produces glycerin to improve skin moisture retention (J Invest Dermatol, 1960, 34:171-174), synthesizes organic acids to maintain an acidic condition on the skin (Br J Dermatol 1986, 80:279-281), and produces antibiotic substances to inhibit the growth of harmful microbes such as Staphylococcus aureus on the skin (N Engl J Med, 1998, 339:520-532; Nature, 2010, 465:346-349; Peptides, 2010, 31:1661-1668; J Invest Dermatol, 2010, 130:192-200), thus exhibiting beneficial effects on the skin.

Examples of the harmful skin microbes include, but are not limited to, Staphylococcus aureus, Cutibacterium acnes, and the like.

In particular, Staphylococcus aureus is a gram-positive facultative anaerobe, which is commonly found on the skin or nasal lining of healthy people and animals. Staphylococcus aureus is known to be the causative bacteria of foodborne illness as well as pyogenic diseases, such as skin abscesses, middle ear inflammation, bladder inflammation, and the like.

Cutibacterium acnes secretes lipases that break down triglycerides in the sebum to form free fatty acids and stimulate hair follicles. Also, it is known that an immunological response, etc. to this bacterium is involved in the formation of acne.

In the present invention, the term ‘reforming’ refers to a state in which the composition of microbial flora undergoes a change in a direction that benefits the human body due to administration of a composition. Said term may specifically refer to promoting the growth of skin-resident beneficial microbes while inhibiting harmful microbes, to thereby increase the proportion of beneficial microbes in skin microbial flora.

One aspect of the present invention provides a composition for reforming skin microbial flora, the composition including: a ginseng extract; a ginseng-derived antibacterial agent; and an alkanediol.

The composition for reforming skin microbial flora of the present invention may promote the growth of beneficial microbes while inhibiting the growth of harmful microbes. Increase in the proportion of beneficial microbes in microbial flora may serve to prevent infections by pathogenic microorganisms, enhance immunity, improve the skin health, and reduce body odor.

The ginseng extract may be obtained from a ginseng by a known technique. For the ginseng, any ginseng that is cultivated or commercially available may be used, without limitations. The ginseng may include various processed types and may be, for example, at least one selected from the group consisting of fresh ginseng, ginseng root hair, black ginseng, wild ginseng, cultivated wild ginseng, mountain-cultivated ginseng, dextrinized ginseng, enzymatically-treated ginseng root hair, fermented ginseng, red ginseng, and fermented red ginseng, but is not limited thereto. The fresh ginseng refers to a raw ginseng retaining its original form, and the ginseng root hair refers to fine roots of ginseng. The red ginseng is a ginseng which is prepared by steaming and drying a raw ginseng, and which exhibits a yellowish brown to reddish brown color due to browning reactions during the process of drying. The black ginseng refers to a ginseng which is prepared by steaming and drying red ginseng several more times, and which as a result has turned black in color. The wild ginseng refers to a naturally-growing ginseng, and the cultivated wild ginseng refers to a wild ginseng planted and cultivated. The mountain-cultivated ginseng refers to a ginseng cultivated by artificially sowing of seeds or transplantation of ginseng seeding in forestlands in the mountains.

As an extraction method for obtaining an extract from the ginseng above, any conventional and known extraction method may be used such as a solvent extraction method, an ultrasonic extraction method, a filtration method, a reflux extraction method, etc., and preferably, the extract may be prepared using a solvent extraction method or a reflux extraction method. Such an extraction process may be repeated several times and may be subsequently followed by additional steps such as concentration and freeze-drying. In particular, the extract thus obtained may be concentrated under reduced pressure to produce a concentrate. The concentrate may be freeze-dried, and using a grinder, high-concentration extract powder may be prepared therefrom. The extract includes a fraction obtained by further fractionation of the extract.

When obtaining an extract from the ginseng, one or more selected from the group consisting of water, an organic solvent, supercritical fluid, and mixtures thereof, may be used as an extraction solvent. The organic solvent may be any one selected from the group consisting of an alcohol, preferably a lower alcohol of C1-C4, hexane (n-hexane), ether, glycerol, propylene glycol, butylene glycol, ethyl acetate, methyl acetate, dichloromethane, chloroform, ethyl acetate, benzene, and a mixed solvent thereof, and may be preferably ethanol. For example, when a mixture of water and an organic solvent is used as the extraction solvent, the mixture of water and an organic solvent may be preferably a mixture of water and a lower alcohol of C1-C4, and more preferably, a mixture of water and ethanol.

In the present invention, the ginseng-derived antibacterial agent may be a purified fraction of an extract of ginseng of ethanol or an aqueous ethanol solution using a column. The fraction may be, but is not limited to, a form of solid powder obtained by performing additional steps such as concentration and/or freeze-drying, etc.

Regarding the preparation of the ginseng-derived antibacterial agent, details on the ginseng and the process for obtaining an extract therefrom may be the same as described above, and therefore will not be further described in details.

The aqueous ethanol solution used in preparing the ginseng-derived antibacterial agent may be at least 50% (v/v) and less than 100% (v/v), and may be 50 to 95% (v/v), 50 to 90% (v/v), and 50 to 85% (v/v), but is not limited thereto. When the concentration of the aqueous ethanol solution is less than 50% (v/v), antibacterial effects may be relatively lower compared to when extraction is performed with an appropriate ethanol content.

For a stationary phase to use in preparing the purified fraction, a filler such as silica gel, activated alumina, synthetic polymers, magnesium silicate, activated carbon, cellulose, ion exchange polymers, etc. may be used, and it is preferable to use silica gel or a synthetic aromatic polymer as the filler. When using a synthetic aromatic polymer, the column may be an aromatic-type synthetic adsorbent column, and the filler may be preferably Diaion HP-20 synthetic adsorbent, but is not limited thereto. Separation using such a column may be performed from once to several times until a fraction of desired purity is obtained, and as necessary, concentration and recrystallization may be performed.

A desorption solvent for preparing the above purified fraction may be any one selected from the group consisting of water, an organic solvent, and mixtures thereof. The organic solvent may be any one selected from the group consisting of alcohols, hexane (n-hexane), ether, glycerol, propylene glycol, butylene glycol, ethyl acetate, methyl acetate, dichloromethane, chloroform, ethyl acetate, benzene, acetone, acetonitrile, and mixed solvents thereof. The organic solvent may be preferably any one selected from the group consisting of acetone, acetonitrile, alcohols of C1-C4, and mixed solvents thereof. The organic solvent may be more preferably ethanol, but is not limited thereto.

When the desorption solvent for preparing the purified fraction is ethanol, the desorption solvent may be ethanol or an aqueous ethanol solution, and the desorption solvent may be, but is not limited to, an aqueous ethanol solution of at least 50% (v/v) and less than 100% (v/v), preferably an aqueous ethanol solution of 50 to 99% (v/v), an aqueous ethanol solution of 70 to 97% (v/v), or an aqueous ethanol solution of 85 to 95% (v/v). When the concentration of the aqueous ethanol solution used as the desorption solvent is less than a lower limit value, desorption of active ingredients to be obtained may take place to an insufficient degree, thus diminishing an antibacterial effect of the fraction.

When preparing the purified fraction, addition of an aqueous ethanol solution, which is the desorption solvent, may be accomplished by passing the aqueous ethanol solution through a column, for example, passing an aqueous ethanol solution through a column under pressure.

The alkanediol used in the present invention may be a colorless and odorless liquid and have a purity of 99.5% or higher. In particular, the alkanediol may be one or more selected from the group consisting of 1,2-butanediol, 1,3-butanediol, 1,4-butanediol, 1,5-pentanediol, neopentyl glycol, isoprene glycol (3-methyl-1,3-butanediol), 1,2-hexanediol, 1,6-hexanediol, 3-methyl-1,5-pentanediol, 1,2-octanediol, 2,3-octanediol, 2-ethyl-1,3-hexanediol, 2-butyl-2-ethyl-1,3-propanediol, 2,5-dimethyl-2,5-hexanediol, 1,2-decandiol, 1,2-dodecandiol, 1,2-tetradecandiol, 1,2-hexadecandiol, 1,2-octadecandiol, 1,12-octadecandiol, 1,2-cyclohexanediol, 1,4-cyclohexanediol, and 1,4-cyclohexane dimethanol, and may be preferably 1,2-hexanediol.

The composition for reforming skin microbial flora of the present invention may include 0.05 to 8 parts by weight of a ginseng extract; 0.01 to 1 part by weight of a ginseng-derived antibacterial agent; and 0.1 to 10 parts by weight of an alkanediol.

For example, the ginseng extract may be 0.05 to 7 parts by weight, 0.07 to 5 parts by weight, 0.1 to 3 parts by weight, or 0.1 to 1 parts by weight, the ginseng-derived antibacterial agent may be 0.03 to 0.7 parts by weight, 0.05 to 0.5 parts by weight, or 0.1 to 0.3 parts by weight, and the alkanediol may be 0.1 to 7 parts by weight, 0.3 to 5 parts by weight, 0.5 to 3 parts by weight, or 0.5 to 1.5 parts by weight.

When included in such amounts, the effect of promoting the growth of beneficial microbes and inhibiting the growth of harmful microbes may be desirably high, thus remarkably improving the increase in beneficial microbes versus harmful microbes.

The composition for reforming skin microbial flora of the present invention may be a cosmetic composition.

The cosmetic composition of the present invention may be prepared in a liquid or solid form, using a base, aids, and additives commonly used in the cosmetics industry. Examples of such cosmetic products in a liquid or solid form may include a skin toner, a cream preparation, a lotion preparation, a bath preparation, and the like, but are not limited thereto. The base, aids and additives commonly used in the cosmetics industry are not particularly limited and may include, for example, water, alcohols, propylene glycol, stearic acid, glycerol, cetyl alcohol, liquid paraffin, and the like.

The cosmetic composition of the present invention may include ingredients commonly used in cosmetic compositions, and for example, may include a carrier and common aids, such as an antioxidant, a stabilizer, a solubilizer, a vitamin, a pigment, and a perfume.

The cosmetic composition of the present invention may be prepared in any formulation commonly produced in the relevant technical field, and for example, may be formulated in, but is not limited to, a solution, a suspension, an emulsion, a paste, a gel, a cream, a lotion, powder, a soap, a surfactant-containing cleanser, an oil, microcapsules, microgranules, an ionic vesicle dispersion, a non-ionic vesicle dispersion, a powder foundation, an emulsion foundation, a wax foundation, a stick, a facial mask, a spray, and the like. More specifically, the cosmetic composition may be prepared in a formulation such as a skin softener, a nutrient skin toner, a nutrient cream, a massage cream, an essence, an eye cream, a cleansing cream, a cleansing foam, cleansing water, a facial pack, spray, and powder.

The cosmetic composition of the present invention, when its formulation is a paste, a cream or a gel, may use animal oils, vegetable oils, wax, paraffin, starch, tragacanth, cellulose derivatives, polyethylene glycol, silicone, bentonite, silica, talc, zinc oxide, or the like, as a carrier component.

The cosmetic composition of the present invention, when its formulation is powder or spray, may use lactose, talc, silica, aluminum hydroxide, calcium silicate, or polyamide powder, as a carrier component. In particular, the cosmetic composition, when its formulation is spray, may further include a propellant such as chlorofluorohydrocarbon, propane/butane, and dimethyl ether.

The cosmetic composition of the present invention, when its formulation is a solution or an emulsion, may utilize a solvent, a solubilizer, or an emulsifier as a carrier component, such as water, ethanol, isopropanol, ethyl carbonate, ethyl acetate, benzyl alcohol, benzyl benzoate, propylene glycol, 1,3-butylene glycol oil, glycerol fatty acid esters, polyethylene glycol, and sorbitan fatty acid esters.

The cosmetic composition of the present invention, when its formulation is a suspension, may utilize, as a carrier component, a liquid diluent such as water, ethanol, and propylene glycol, a suspending agent such as ethoxylated isostearyl alcohol, polyoxyethylene sorbitol esters, and polyoxyethylene sorbitan esters, or microcrystalline cellulose, aluminum methahydroxide, bentonite, agar, or tragacanth, etc.

The cosmetic composition of the present invention, when its formulation is a surfactant-containing cleanser, may utilize, as a carrier component, an aliphatic alcohol sulfate, an aliphatic alcohol ether sulfate, a sulfosuccinic acid monoester, an imidazolinium derivative, methyl taurate, a sarcosinate, a fatty acid amide ether sulfate, alkylamidobetaine, an aliphatic alcohol, fatty acid glycerides, a fatty acid diethanolamide, vegetable oils, a lanolin derivative, an ethoxylated glycerol fatty acid ester, or the like.

The cosmetic composition of the present invention may be used in a single application or in repeated applications, or may be used in repeated applications with another cosmetic composition other than the present invention. In addition, the cosmetic composition of the present invention may be utilized by a common method of use and may be utilized with a frequency that varies depending on a user's skin condition or preference.

The cosmetic composition of the present invention, when its formulation is a soap, a surfactant-containing cleanser, or a surfactant-free cleanser, may be wiped off, detached, or rinsed with water after applied on skin. As specific examples, the soap may include, but is not limited to, a liquid soap, a powder soap, a solid soap, or an oil soap, the surfactant-containing cleanser formulation may be, but is not limited to, a cleansing foam, a cleansing solution, a cleansing cloth, and a cleansing pack, and the surfactant-free cleanser formulation may be, but is not limited to, a cleansing cream, a cleansing lotion, a cleansing solution, or a cleansing gel.

### MODE FOR CARRYING OUT THE INVENTION

Herein below, the present invention is described in greater detail through Preparation Examples and Examples.

However, the following Preparation Examples and Examples are provided only to illustrate the present invention, and therefore, the content of the present invention should not be limited to the following Preparation Examples and Examples.

### EXAMPLE 1

### 1-1. Preparation of Red Ginseng Concentrate

1 kg of 6 year-old red ginseng was combined with a 6× volume of purified water, and was extracted at room temperature over 8 hours and then concentrated under reduced pressure, to produce a red ginseng concentrate.

### 1-2. Preparation of Red Ginseng Antibacterial Powder

1 kg of red ginseng was combined with a 5.5× volume of 70% (v/v) ethanol and was extracted 3 times at 70° C. over 8 hours. Using a rotary evaporator on the resulting extract, a red ginseng alcohol-extract concentrate having a water content of 40% was obtained in an amount of 50 g or more. The red ginseng alcohol-extract concentrate was combined and diluted with a 3× volume of purified water, and 150 g of the resulting diluted solution was passed through a column filled with 100 g of HP-20 ion-exchange polymer. After rinsing the column with purified water of a volume 5 times the volume of the diluted solution passed, an aqueous ethanol solution (95% (v/v)) was passed through the column, and an active ingredient fraction attached to the ion-exchange polymer column was collected. After completely evaporating solvents from the fraction thus obtained, the fraction was combined with purified water and frozen at −70° C. and then dried in a freeze-dryer, to produce solids.

### 1-3. Preparation of Composition for Reforming Skin Microbial Flora

0.1 g of the red ginseng concentrate and 0.01 g of the red ginseng antibacterial powder were dissolved in 10 ml of a saline solution and then mixed with 0.1 ml of 1,2-hexanediol, to produce the composition for reforming skin microbial flora of Example 1.

### COMPARATIVE EXAMPLES 1 to 5

Composition for reforming skin microbial flora was prepared by the same method as above, but with changed ingredients in the composition for reforming skin microbial flora as shown in the table below.

TABLE 1

| Number | Blending conditions |
|---|---|
| Example 1 | Red ginseng concentrate (10 mg/ml) + red ginseng |
| | antibacterial powder (1 mg/ml) + hexanediol (1%) |
| Comparative | Red ginseng concentrate (10 mg/ml) |
| Example 1 | |
| Comparative | 3-Sialyllactose (10 mg/ml) |
| Example 2 | |
| Comparative | 6-Sialyllactose (10 mg/ml) |
| Example 3 | |
| Comparative | Red ginseng concentrate (10 mg/ml) + anisic acid |
| Example 4 | (2 mg/ml) |
| Comparative | Red ginseng concentrate (10 mg/ml) + red ginseng |
| Example 5 | antibacterial powder (1 mg/ml) |

### EXPERIMENTAL EXAMPLE 1

To compare fructooligosaccharides and the composition of Example in terms of the effect of promoting the growth of beneficial skin microbes, samples were treated respectively, and changes in absorbance by beneficial skin microbes were measured.

In particular, the red ginseng concentrate of Preparation Example 1 and fructooligosaccharides (purity of 95% or higher) (purchased from Yungjin Pharm) were each dissolved in a saline solution at concentrations of 0.078 mg/ml, 0.156 mg/ml, 0.313 mg/ml, 0.625 mg/ml, 1.25 mg/ml, 2.5 mg/ml, 5 mg/ml, and 10 mg/ml.

Prepared samples were inoculated with Staphylococcus epidermidis (Strain number 1917, Korean Collection for Type Cultures) and incubated over 24 hours in a shaking incubator.

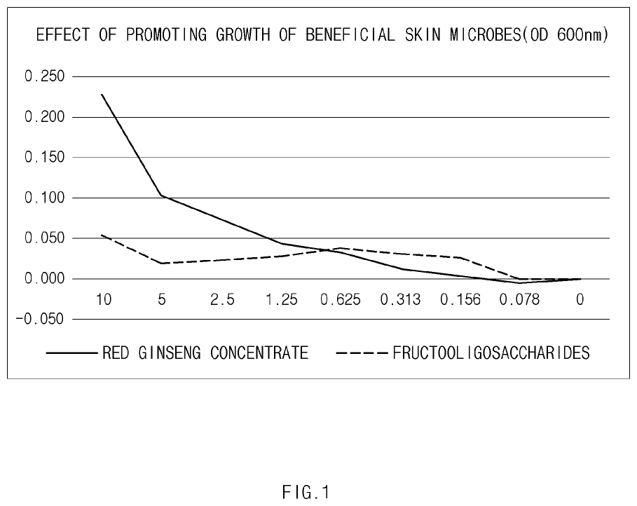

Solution with the strain grown therein was collected and centrifuged, and after removing the supernatant therefrom, an equivalent amount of a saline solution was added to prepare a bacterial solution. Then, absorbance at 600 nm was measured, and the result thereof is shown in FIG. 1.

From the result shown in FIG. 1, it was confirmed that compared to the fructooligosaccharides, which is known to be prebiotics, the red ginseng concentrate exhibits a far superior growth promoting effect even at a low concentration of 0.625 mg/ml.

### EXPERIMENTAL EXAMPLE 2

To compare the compositions of Example and Comparative Examples in terms of the increase in growth of beneficial skin microbes versus harmful skin microbes, the growth of each microbe was measured by the same method as described in Experimental Example 1.

Specifically, using Staphylococcus epidermidis as beneficial skin microbes, and Staphylococcus aureus (Strain number 39149, Korean Collection for Type Cultures) as harmful skin microbes, samples were inoculated respectively and incubated for 24 hours in a shaking incubator.

Solutions with the strains grown therein were each collected and centrifuged, and after removing the supernatant therefrom, an equivalent amount of a saline solution was added to prepare a bacterial solution. Then, absorbances at 600 nm were measured, and the result thereof is shown in Table 2 below.

TABLE 2

| | OD600 (Beneficial | Increase in beneficial |
|---|---|---|
| | microbes/Harmful | microbes versus harmful |
| Number | microbes) | microbes |
| Example 1 | 0.713/0.121 | ×5.893 |
| Comparative Example 1 | 0.364/0.902 | ×0.403 |
| Comparative Example 2 | 0.049/0.077 | ×0.636 |
| Comparative Example 3 | 0.047/0.065 | ×0.723 |
| Comparative Example 4 | 0.173/0.928 | ×0.186 |
| Comparative Example 5 | 0.217/0.332 | ×0.653 |

From the result shown in Table 2 above, it was found that the composition for reforming skin microbial flora of the present invention selectively activates only the beneficial microbes rather than the harmful microbes. It was also found that this selective growth-promoting effect is significantly higher than when treated with the red ginseng concentrate alone (Comparative Example 1) or when treated with a combination of the red ginseng concentrate and the red ginseng antibacterial powder (Comparative Example 5).

In addition, compared to the composition of the present invention, when treated with sialyllactose (Comparative Examples 2 and 3), which is one of human milk oligosaccharides (HMO) present with the highest concentration in colostrum of breast milk, the effect of promoting the growth of beneficial microbes was negligible, and the effect of selectively promoting only beneficial microbes was also low.

Likewise, when the red ginseng concentrate was combined with anisic acid (Evonik Dr. Straetmans GmbH, Germany) (Comparative Example 4), the effect of selectively promoting the growth of beneficial microbes alone was negligible compared to Example of the present invention.

### PREPARATION EXAMPLE 1

### 1-1. Preparation of Essence

Using a combination of the red ginseng concentrate, red ginseng antibacterial powder, and hexanediol of the present invention, an essence was prepared according to the contents (parts by weight) shown in Table 3 below.

TABLE 3

| Ingredient | Content (Parts by weight) |
|---|---|
| Triethanolamine | 0.25 |
| Carboxy vinyl polymer | 0.22 |
| Glycerin | 4 |
| Butylene glycol | 2 |
| Red ginseng concentrate of Example 1-1 | 5 |
| Purified fraction of Example 1-2 | 0.5 |
| Hexanediol | 1 |
| Beeswax | 0.5 |
| Cetostearyl alcohol | 1 |
| Glyceryl monostearate | 1 |
| Squalene | 4 |
| Purified water | to 100 |

### 1-2. Preparation of Skin Softener

Using a combination of the red ginseng concentrate, red ginseng antibacterial powder, and hexanediol of the present invention, a skin softener was prepared according to the contents (parts by weight) shown in Table 4 below.

TABLE 4

| Ingredient | Content (Parts by weight) |
|---|---|
| 1,3-butylene glycol | 1.00 |
| Disodium EDTA | 0.05 |
| Allantoin | 0.10 |
| Dipotassium glycyrrhizate | 0.05 |
| Citric acid | 0.01 |
| Sodium citrate | 0.02 |
| Glycereth-26 | 1.00 |
| Arbutin | 2.00 |
| PEG-40 hydrogenated castor oil | 1.00 |
| Ethanol | 30.00 |
| Red ginseng concentrate of Example 1-1 | 5 |
| Purified fraction of Example 1-2 | 0.5 |
| Hexanediol | 1 |
| Colorant | Trace amount |
| Fragrance | Trace amount |
| Purified water | to 100 |

### 1-3. Preparation of Nutrient Cream

Using a combination of the red ginseng concentrate, red ginseng antibacterial powder, and hexanediol of the present invention, a nutrient cream was prepared according to the contents (parts by weight) shown in Table 5 below.

TABLE 5

| Ingredient | Content (parts by weight) |
|---|---|
| 1,3-butylene glycol | 7.0 |
| Glycerin | 1.0 |
| D-panthenol | 0.1 |
| Magnesium aluminum silicate | 0.3 |
| PEG-40 stearate | 1.2 |
| Stearic acid | 2.0 |
| Polysorbate 60 | 1.5 |
| Lipophilic glyceryl stearate | 2.0 |
| Sorbitan sesquioleate | 1.5 |
| Cetearyl alcohol | 3.0 |
| Mineral oils | 4.0 |
| Squalene | 3.8 |
| Red ginseng concentrate of Example 1-1 | 5 |
| Purified fraction of Example 1-2 | 0.5 |
| Hexanediol | 1 |
| Vegetable oils | 1.8 |
| Dimethicone | 0.4 |
| Dipotassium glycyrrhizate | Trace amount |
| Allantoin | Trace amount |
| Sodium hyaluronate | Trace amount |
| Tocopheryl acetate | Trace amount |
| Triethanolamine | Trace amount |
| Fragrance | Trace amount |
| Purified water | to 100 |

### 1-4. Preparation of Lotion

Using a combination of the red ginseng concentrate, red ginseng antibacterial powder, and hexanediol of the present invention, a lotion was prepared according to the contents (parts by weight) shown in Table 6 below.

TABLE 6

| Ingredient | Content (parts by weight) |
|---|---|
| Cetostearyl alcohol | 1.6 |
| Stearic acid | 1.4 |
| Lipophilic glyceryl monostearate | 1.8 |
| PEG-100 stearate | 2.6 |
| Sorbitan sesquioleate | 0.6 |
| Squalene | 4.8 |
| Macadamia nut oil | 2 |
| Jojoba oil | 2 |
| Tocopherol acetate | 0.4 |
| Methyl polysiloxane | 0.2 |
| Tocopherol acetate | 0.4 |
| 1,3-butylene glycol | 4 |
| Xanthan gum | 0.1 |
| Glycerin | 4 |
| D-panthenol | 0.15 |
| Red ginseng concentrate of Example 1-1 | 5 |
| Purified fraction of Example 1-2 | 0.5 |
| Hexanediol | 1 |
| Allantoin | 0.1 |
| Carbomer (2% aq. Sol) | 4 |
| Triethanolamine | 0.15 |
| Ethanol | 3 |
| Purified water | to 100 |

1. A composition for reforming skin microbial flora, the composition comprising: a ginseng extract; a ginseng-derived antibacterial agent; and an alkanediol.

2. The composition of claim 1, wherein the ginseng-derived antibacterial agent is a column purified fraction of ethanol extract of ginseng, or aqueous ethanol solution extract of ginseng with an ethanol content of at least 50% (v/v) to less than 100% (v/v).

3. The composition of claim 2, wherein stationary phase of the column is an aromatic synthetic polymer.

4. The composition of claim 2, wherein desorption solvent for preparing the purified fraction is ethanol or an aqueous ethanol solution with an ethanol content of at least 50% (v/v) to less than 100% (v/v).

5. The composition of claim 1, wherein the ginseng extract is extracted with one or more solvent selected from the group consisting of water and alcohols having 1-4 carbon atoms.

6. The composition of claim 1, wherein the alkanediol is 1,2-hexanediol.

7. The composition of claim 1, comprising: 0.05 parts by weight to 8 parts by weight of the ginseng extract; 0.01 parts by weight to 1 part by weight of the ginseng-derived antibacterial agent; and 0.1 parts by weight to 10 parts by weight of the alkanediol.

8. The composition of claim 1, wherein the reforming of skin microbial flora promotes the growth of beneficial skin microbe and inhibits the growth of harmful skin microbes.

9. The composition of claim 8, wherein the beneficial skin microbes comprise one or more selected from the group consisting of Staphylococcus epidermidis, Streptococcus pneumonia, Lactobacillus acidophilus, Lactobacillus Rhamnosus, and Bifidobacterium bifidum.

10. The composition of claim 8, wherein the harmful skin microbes comprise one or more selected from the group consisting of Staphylococcus aureus and Cutibacterium acnes.

11. The composition of claim 1, wherein the composition is a cosmetic composition.

12. The composition of claim 11, wherein the cosmetic composition is a formulation selected from the group consisting of a solution, a suspension, an emulsion, a paste, a gel, a cream, a lotion, a powder, a soap, a surfactant-containing cleanser, an oil, microcapsules, microgranules, an ionic vesicle dispersion, a non-ionic vesicle dispersion, a powder foundation, an emulsion foundation, a wax foundation, a stick, a facial mask, and a spray.
